(19) United States
(12) Patent Application Publication
(10) Pub. No.: US 2005/0038492 A1
(43) Pub. Date: Feb. 17, 2005

(54) Method for forming matrices of hardened material

(76) Inventors: Christopher Mason, London (GB); Martin Arthur Town, London (GB)

Correspondence Address: NIXON & VANDERHYE, PC, 1100 N GLEBE ROAD, 8TH FLOOR, ARLINGTON, VA, 22201-4714, US

(21) Appl. No.: 10/496,840

(22) PCT Filed: Dec. 4, 2002
(86) PCT No.: PCT/GB02/05475

(30) Foreign Application Priority Data
Dec. 4, 2001 (GB) 0129008.9

(51) Int. Cl. B29C 67/20; A61F 2/06; B29D 1/00; A61F 9/00; A61F 11/00

(52) U.S. Cl. 623/1.1; 264/49; 623/901; 623/921

(57) ABSTRACT

A matrix of hardened material, typically biocompatible material, is formed by contacting a hardenable liquid with a volume blanking structure, the structure having a dispersion of interconnected spaces and including a hardening agent and allowing the hardenable liquid to harden by interaction with the hardening agent to form the matrix. The volume blanking structure may be removed to leave corresponding voids in the matrix of hardened material. The hardenable liquid may contain viable cells.

### FIELD OF THE INVENTION

The present invention relates to the production of hardened material in the form of a matrix, by hardening a hardenable liquid. The invention is particularly applicable to the production of bulk matrices of biocompatible material, but is not limited to this.

### DESCRIPTION OF THE PRIOR ART

There is a demand for replacement tissue for implantation into the human body. Typically, replacement blood vessels are required for use in surgical procedures (Vacanti and Langer (1999), Lancet 35A, Supplement 1: pages 32 to 34). Also, there is a need for replacement tissue of bulkier and/or more complex nature, such as body organs. Typical examples include organs such as the liver, kidneys and heart.

In the case, for example, of dual kidney failure, a patient faces the prospect of artificial dialysis until a suitable donor kidney is found for transplantation. Dialysis has the drawbacks of inconvenience and the risk of infection. Transplantation is often complicated by rejection problems. Even more severe complications are presented when the organ which fails is the heart or liver.

There have been attempts to construct tissue engineering scaffolds using biocompatible materials. Typical scaffold materials are plastics materials such as PGA (polyglycolic acid), PLA (polylactic acid) and PLGA (polylactic coglycolic acid). These materials are formed into desired shapes by conventional techniques such as melting followed by extrusion or moulding. Since the plastics material must be melted before extrusion or moulding, there is limited opportunity to incorporate biologically active molecules or cells in the shaped material.

Previous attempts to construct tissue engineering scaffolds have involved the seeding and growth of cells on sheets or tubes of biocompatible material. Attempts to make bulk tissue engineering scaffolds have involved lamination of sheets such as PGA to give a thicker construction (see, for example, Mikos A G, Sarakinos G, Leite S M, Vacanti J P, and Langer R, Laminated three dimensional biodegradable foams for use in tissue engineering, 1993 BIOMATERIALS 14; 323-330, and Cima L G and Cima M J, 1996, Tissue regeneration matrices by solid free-form fabrication techniques, U.S. Pat. No. 5,518,680). However, such techniques are complex and involve many steps of lamination. This makes these techniques unsuited to anything more than laboratory scale experimental use.

It is also known to encapsulate cells in materials such as alginate for implantation into mammals, in order to achieve delivery of therapeutic molecules secreted by the cells to a desired tissue (see for example T. A. Read et al, Nature Biotechnology 19, pages 29 to 34 and T. Joki et al, Nature Biotechnology 19, pages 35 to 39). In this case, cells are typically encapsulated in beads of alginate.

It is also known to mix cells with collagen and allow the mixture to set. However, FDA-approved collagen is extremely costly (around $1 per microgram) so this technique is unsuited to the formation of tissue engineering scaffolds of useful size.

WO-00/62829 describes manufacture of biocompatible porous polymer scaffolds by pouring a solution of polymer in two miscible solvents onto water-soluble particles, then cooling to crystallise the polymer and removing the solvents and the particles.

### SUMMARY OF THE INVENTION

Accordingly, in a first aspect, the present invention provides a method of forming a matrix of hardened material, including the steps of:
contacting a hardenable liquid with a volume blanking structure, the structure having a dispersion of interconnected spaces therein and including a hardening agent, whereby the hardenable liquid occupies at least some of said spaces in said structure; and
allowing the hardenable liquid to harden by interaction with the hardening agent to form the matrix.

By “hardened material” is meant a material which is sufficiently hard substantially to retain its form or shape without the volume blanking arrangement, but it may sag to some extent. Matrices formed according to the present invention will typically be flexible and preferably will be resilient. Indeed, matrices formed according to this invention may be delicate. Thus the term “hardened material” is used to encompass, inter alia, materials having a high liquid content such as hydrogels and readily deformable and flexible materials. However, the formation of more rigid structures is also contemplated.

In this first aspect, the invention may be envisaged as providing a “negative” or mould for the final hardened matrix by the volume blanking structure. The volume blanking structure may be envisaged as “blanking out” certain volumes to the hardenable liquid, i.e. excluding the hardenable liquid from those volumes. Thus, the hardenable liquid is locatable in the interconnected spaces but is excluded from the blanked-out volumes.

It is not necessary that the volume blanking structure is formed before contact is made with the hardenable liquid, although this is preferred.

Typically, the hardening interaction is a chemical interaction, such as cross-linking of molecules in the hardenable liquid. By a chemical interaction is meant typically a chemical reaction causing chemical change. A physical change only, e.g. crystallisation such as the crystallisation procedure of WO-00/62829, does not constitute a chemical interaction. A suitable combination of hardenable liquid and hardening agent can be chosen depending on the use to which the structure to be formed is to be put, and taking account of constraints imposed by other components to be incorporated within the structure.

The method may further include the step of removing the volume blanking structure to leave corresponding voids in the matrix of hardened material. Preferably, the volume blanking structure is removed by dissolving it. The matrix of hardenable material remaining is preferably porous.

In the case where the matrix has a distribution of voids in it after removal of the volume blanking structure, the voids are preferably interconnected. This allows fluid (e.g. cell culture medium) to flow through the matrix, via the interconnected voids.

It is preferred that the matrix of hardened material is a bulk matrix. This is in contrast with the hardenable liquid being hardened into the form of a sheet or a tube. Preferably, the bulk matrix has a three dimensional shape whose smallest external overall dimension is not less than 0.5 mm, 1 mm, 2 mm, 5 mm or, more preferably 10 mm. Forming a bulk matrix has the advantage that a tissue engineering scaffold can be formed essentially in one piece, rather than by layering of individual thin pieces of material. As is explained later, an advantage of embodiments of the present invention can be that the overall shapes of the matrices formed can be complex.

The method may include the step of distributing or seeding a bioactive agent such as cells in the matrix. This may be performed after the volume blanking arrangement is removed, for example by inserting the bioactive agent into the voids left by the removed volume blanking arrangement. However, preferably, bioactive agent, particularly cells, are seeded in the matrix by mixing the cells with the hardenable liquid before it hardens, for example before the liquid is contacted with the volume blanking structure. If the matrix is to be a tissue engineering scaffold, it may be advantageous to include a cell growth factor. Typically, this may be included as part of the volume blanking structure, which transfers or is transferred to the matrix before removal of the volume blanking structure.

Advantageously, the hardened material is a biocompatible material. A biocompatible material is considered to be any material which is not excessively harmful or toxic to living cells or tissue, i.e. non-toxic in the intended environment of use. The material may be inert, or may be degradable by living cells or tissue, for example by enzymes produced by living cells or tissue. The material may be suitable for direct implant to a mammalian body. The biocompatible material may be suitable for use as a scaffold for growth of cells either on or within the material as mentioned above. Suitable materials include biologically derived substances such as alginate, collagen, etc., and synthetic materials such as heat-softening materials or thermoplastics, etc. Preferably, these will be inert, or will not give rise to excessively toxic degradation products.

Suitable hardened materials may have a structure which allows the controlled release of bioactive agents or substances such as pharmaceuticals, hormones, growth factors, cytokines, antibodies, nucleic acids such as DNA, isolated cell organelles such as mitochondria, killed cells, and the like.

Additionally or alternatively, living cells may be encapsulated in the matrix of hardened material. These cells may be eukaryotic or prokaryotic. In this case the matrix may support exchange of proteins, nutrients, oxygen, secreted molecules and waste products between the cells and a medium surrounding and/or penetrating the hardened matrix.

In this way, the hardened material may act as a tissue engineering scaffold, supporting growth of the cells. Structures containing living cells may be cultured in vitro or implanted directly into a patient. When cultured in vitro, the scaffold may be degraded when the cells have formed an integral mass (e.g. cells and extra cellular matrix) or body and when physical support from the scaffold is no longer required. Degradation may be by auto-degradation, or may be caused by a degradative agent such as an enzyme, which may be added exogenously or produced by the cells within the structure. For example, alginate matrix can be degraded by exposure to sodium ions or by lyases. An alternative method of degradation could involve using antibodies or antibody fragments. The hardenable material may be chosen appropriately, depending on the intended use, e.g. whether the scaffold is to be degraded prior to implantation or not.

Suitable combinations of hardenable liquids and hardening agents are well known in the art. For example, alginate, e.g. sodium alginate can be cross-linked by calcium ions into a suitable biocompatible material. Accordingly, the hardenable liquid may contain sodium alginate, and may be hardened by contact with a calcium salt, such as calcium chloride, as the hardening agent. Another possible combination of components includes acid soluble collagen which cross-links to form a hydrogel when exposed to sodium hydroxide and/or when heated to a temperature of above around 4° C. up to around 37° C. Another possible combination is a mixture of fibronectin and fibrinogen dissolved in urea which forms a solid when exposed to a solution of hydrochloric acid/calcium chloride. In general terms, any natural or synthetic polymers which, for example, are cross-linkable and which are biocompatible (preferably also during cross-linking or polymerisation) may be used. Combinations of hardenable liquids may be used, e.g. a mixture of alginate and collagen.

The hardenable liquid may include structurally modified molecules. For example, alginate may be used where the alginate is modified to include a peptide, e.g. a pentapeptide including for example an RGD sequence attached to the alginate molecules in order to provide cell attachment locations.

The hardening agent is not limited to chemical compounds, although this may be preferred. The hardening agent may, for example, bring about a temperature change in the hardenable liquid, e.g. it may heat the hardenable liquid to bring about a chemical alteration in the hardenable liquid (e.g. polymerisation or cross-linking).

The hardenable liquid may further contain one or more biologically active agents. These active agents may be active molecules such as enzymes, growth factors, hormones, cytokines, antibodies, nucleic acids, killed cells, isolated cellular organelles, etc. Additionally or alternatively, the biologically active agents may include live cells. If the hardened matrix is required to contain a uniform distribution of a biologically active agent, then the active agent may be homogeneously mixed into the hardenable liquid, consequently being uniformly distributed through the structure of the resultant hardened matrix.

The volume blanking structure may be an arrangement or structure formed of one or more (preferably more than one) volume blanking elements. In this preferred case, the interconnected spaces may be interstices between adjacent volume blanking elements. Typically, these elements are packed so that at least some adjacent elements touch. This packing may be in a suitable vessel such as a tube. In this case, the packed elements may be supported in the tube by a removable sealing member. It is clearly desirable to form the hardened matrix in a vessel in order to contain the hardenable liquid. However, the vessel may also provide an external limit to the shape of the volume blanking arrangement and therefore provide the overall shape of the hardened matrix.

The volume blanking structure is typically solid in the sense that it may be self-supporting. Of course, the volume blanking structure may be further supported by a vessel, such as referred to above, to further support its shape.

The volume blanking elements are typically solid units, but they may alternatively be gaseous or liquid (e.g. bubbles or droplets). They may be hollow. They may be small enough to move relative to each other in the volume blanking structure if disturbed.

Typically, the volume blanking elements have an average size of 500 μm or less or, more preferably, 100 μm or less. This average size is preferably more than 1 μm and even more preferably more than 2 μm. The size distribution of volume blanking elements may be polymodal, e.g. bimodal. For example, there may be an array of larger volume blanking elements with smaller volume blanking elements. This is discussed in more detail below.

Clearly, for matrices of a useful size, there will be very many volume blanking elements used. This will provide very many interconnected spaces into which the hardenable liquid may flow. Advantageously, after removal of the volume blanking elements, the matrix will therefore have a very high internal surface area (i.e. the surface area of the voids).

Preferably, the voids have an average size in the same range as that defined for the volume blanking elements, above. Of course, the voids may change size after removal of the volume blanking elements.

The preferred pourability of the volume blanking elements means that they may be poured into a vessel which can define, in part, an overall shape for the matrix. Thus, the small size of the volume blanking elements means that complex overall shapes, such as the shapes or organs, can be replicated.

The method may be carried out by first mixing the hardenable liquid with the volume blanking elements and then subsequently pouring the mixture into a vessel or mould.

Typically, each volume blanking element may be a bead. Each bead may be spherical or approximately spherical in shape. However, other suitable shapes may be envisaged, typically rounded shapes such as ellipsoidal or pebble-shape. There are known methods for production of beads of the preferred size. Such methods can give beads of narrow size distribution. See, for example, New Approaches to Tablet Manufacture. Dr. Marshall Whiteman, Phoqus. European Pharmaceutical Review, Vol. 4, Issue 3, Autumn 1999, and Cowley M, 1999, Powder Coating: Assessment of component being coated: A practical guide to equipment, processes and productivity at a profit, pp. 13-31.

If, as is preferred, the volume blanking structure is to be removable from the hardened matrix, then this places a constraint on the materials which may be used for the volume blanking structure. Preferably, the material is a solid which is soluble in a biocompatible solvent. It is preferred that the material does not dissolve immediately on contact with the hardenable liquid, since the volume blanking arrangement should give some mechanical integrity to the hardenable liquid as it hardens. A suitable material for the volume blanking structure is a soluble sugar such as glucose. The material of the volume blanking structure may be capable of sublimation. The material may be biological feedstock such as carbohydrate, protein, fat or it may be enzymatically degradable. In this case, the material would be useful for culturing and growing cells which are seeded in the matrix.

The volume blanking structure may include, e.g. collagen, alginate or similar hardened materials. The volume blanking structure may include bone-like materials, such as hydroxyapatite (HA). In that case, the hardened matrix may be a tissue engineering scaffold for bone tissue. Part of the volume blanking structure (e.g. the HA) can then stay within the matrix to become part of the final engineered tissue.

Typically, the hardening agent is formed as a layer on at least some of the volume blanking elements. An advantage here is that the hardening agent will come into contact with the hardenable liquid before the remainder of the volume blanking element.

The hardening agent layer may have a protective layer formed over it, e.g. an enteric layer. This protective layer is adapted to dissolve at a predetermined rate in the hardenable liquid. This can delay the exposure of the hardening agent to the hardenable liquid. In this way, hardening of the hardenable liquid can be delayed up until all of the volume blanking structure has been contacted with hardenable liquid. In a preferred embodiment, the solubility of the protective layer in the hardenable liquid may be dependent on pH. In this way, the dissolution of the protective layer may be triggered by a change in pH of the hardenable liquid.

The volume blanking elements may further include a cell growth factor layer. This may be above or below the hardening agent layer, depending on when in the hardening process it would be suitable for the growth factor to be released. Typically, the cell growth factor layer will be underneath the hardening agent layer, thereby to release the growth factor layer substantially after hardening of the hardenable liquid has occurred.

In some preferred embodiments, the formation of the volume blanking structure includes the formation of one or more selected regions within the arrangement with different concentrations of hardening agent to the remainder of the arrangement. An effect of such concentration of variations can be to affect the hardening of the hardenable liquid in those regions. In order to accurately construct such regions in the arrangement, each selected region may be separated from the remainder of the structure or arrangement by a retaining surface, such as by a soluble film. This allows the volume blanking structure to be formed with accurate distribution of concentration of hardening agent.

Each selected region with different concentration of hardening agent may be an elongate region extending through the structure. Preferably, the concentration of hardening agent in such regions is insufficient to harden the hardenable liquid placed in the interconnected spaces in such regions. An effect of this can be that the matrix includes regions of non-hardened liquid. In the case where this liquid is subsequently removed, the matrix will include non-filled spaces corresponding to these selected regions. In this way, the overall internal shape of the matrix may be controlled by controlling the concentration distribution of hardening agent through the arrangement. These regions can be formed so as to define vessels or chambers within the hardened matrix. In this way, the complex internal shapes of organs such as the liver, kidney, heart, etc. can be mimicked. Of course, in the case of mimickery of such an organ, the matrix may preferably be seeded with suitable cells (for example, cells from such an organ from the patient of interest) and other suitable bioactive substances. Of course, the term “organ” is not limited to these described body parts, but is applicable to other body parts such as skin, bone, body lumens such as blood vessels, parts of the gastro-intestinal tract, etc.

As mentioned above, the volume blanking structure may include a polymodal size distribution of volume blanking elements. Significantly larger (e.g. greater than 1 mm in size) volume blanking elements may be included. Once dissolved away, these would leave large pores in the matrix. Subsequently, these larger pores may be filled (e.g. by injection) with a mixture of hardenable liquid and volume blanking elements. Typically, this method allows a main matrix to be formed and seeded with a first cell type (mixed with the hardenable liquid). Then one or more of the large pores may be filled with matrix seeded with a second cell type (mixed with the injected hardenable liquid). In this way, islets of a second cell type may be formed in a matrix of a first cell type. Of course, this is not limited to two cell types. Three or more may be used. Furthermore, the larger pores may have predetermined shapes, e.g. rod-shaped, dependent on the shapes of the larger volume blanking elements used.

Furthermore, the internal surface of a film used to separate a selected region from the rest of the matrix may be used as a guide surface for the formation of a sheet or preferably a tube of hardened material. Typically this, e.g. tube is seeded with cells of, e.g. smooth muscle type. Typically, the guide surface will be in the form of an internal surface of a tube.

Preferably, the method further includes the steps of:
providing a body of hardenable liquid (e.g. the hardenable liquid described above, or a different one) in contact with a guide surface for the formation of the layer,
relatively moving a regulator member and said guide surface with a gap between them so that a portion of said body of hardenable liquid is exposed on said guide surface as a layer of predetermined thickness thereon,
causing hardening of the layer of hardenable fluid thus formed (e.g. by a hardening agent), to form the hardened layer on the guide surface.

Our published International Patent Application WO-02/77336, claiming priority of UK patent applications 0120815.6 (filed 28 Aug. 2001), 0107549.8 (filed 26 Mar. 2001) and 0121995.5 (filed 11 Sep. 2001), discloses methods of forming hardened sheets and tubes. The entire content of WO-02/77336 is hereby incorporated by reference into the present application, and is referred to below.

For example, the layer of hardenable liquid may be caused to harden by contacting the layer of hardenable liquid with a fluid (hardening agent) causing hardening thereof. The fluid which causes hardening may be selected from:
a gas containing a hardening agent for the hardenable liquid,
a gas effecting hardening of the hardenable liquid by drying,
a liquid comprising a reactive hardening agent, e.g. a cross-linking agent, for the hardenable liquid, and
a liquid effecting hardening of the hardenable liquid by solvent extraction.

Preferably, the fluid causing hardening is progressively immediately contacted with the layer of hardenable liquid as the layer is formed by the relative movement of the regulator member and the guide surface.

The regulator member may act as a barrier separating the fluid causing hardening from said body of the hardenable liquid. Movement of the regulator member may be caused by flow of the fluid causing hardening. The regulator member need not be solid. It may be, for example, gaseous, e.g. a gas bubble sized appropriately.

The regulator member may be driven by a piston action, e.g. by flow of the fluid causing hardening.

The regulator member may a float floating on the hardenable liquid. It may be a gas bubble.

The hardening liquid may comprise a plurality of discrete bands of different solutions, to form a hardened layer having substantially distinct sub-layers.

The hardened layer may be seeded with cells, for example. These cell may be of a different type to those cells (if any) seeded in the matrix. In this way, the matrix of hardened material may be formed with tubes of hardened material extending through it. This is particularly desirable for mimicking the structure of body parts and organs.

The method may further include the step of locating a further shaping insert in the hardened matrix. This may be, for example, by forming the volume blanking structure around one or more inserts having a desirable shape. The inserts may be removable mechanically or by dissolution or by a combination of these (e.g. a mechanically removable skeleton coated with a soluble solid layer).

In a particularly preferred embodiment, the insert or inserts are forked or branched. Particularly, Christmas tree shaped inserts are preferred, i.e. a shape with a main trunk which splits progressively along its length into finer and finer branches (these branches also branching, as appropriate). This may mimic the cardiovascular system. Two (or more) such inserts may be opposed (branched ends facing each other and/or e.g. overlapping and/or intertwining with each other) in a vessel to allow a suitably shaped matrix to be formed.

Matrices provided according to the present invention may be used in a wide variety of ways. In addition to the organ replacement use mentioned above, matrices may be used as structures containing active agents for use in therapeutic devices such as transdermal delivery patches and other therapeutic devices such as tablets or implants or gene therapy delivery devices.

Biocompatible hardened matrices may also be used as internal grafts for delivery of any appropriate active substance directly to an internal organ, or to a disease or wound site. For example, a matrix containing factors for the promotion of wound healing, such as pro-angiogenic factors, may be applied to a section of tissue, such as bowel, to promote knitting together of that tissue after surgery (e.g. surgical anastomosis). Alternatively, pro-angiogenic factors could be delivered to the heart, or anti-angiogenic factors to a tumour in this way.

Accordingly, in a second aspect, the present invention provides a matrix of hardened material obtained or obtainable via the method of the first aspect, including any of the preferred features of the first aspect.

In a third aspect, the present invention provides a matrix of biocompatible in vitro hardened material having an array of interconnected voids therein, the hardened material having a controlled distribution of a bioactive agent within its volume, and wherein the matrix is preferably not a sheet or tube. The matrix material may be hardened by chemical interaction and/or contain cells within the material itself.

Typically, the array of interconnected voids is in the form of a packed structure of contacting rounded shapes, such as spheres. Preferably, the interconnected voids are partially separated from each other by nodes of hardened material, each node having a controlled distribution of the bioactive agent through its thickness.

The bioactive agent may be a pharmaceutical or other bioactive molecule, e.g. a pharmaceutical, enzyme, growth factor, hormone, cytokine, antibody, or nucleic acid, to be delivered to a desired site in a living organism, e.g. mammal. Additionally or alternatively, the bioactive agent may include viable cells, killed cells or isolated cellular organelles.

Preferably, the matrix includes any of the preferred features described with respect to the first aspect.

In a fourth aspect, the present invention provides a tissue growth scaffold including a matrix according to the second or third aspect. Further the invention provides a method of tissue growth, e.g. replacement organ growth, comprising cultivating cells contained in the hardened matrix material and/or cells present in the voids within the matrix.

In a fifth aspect, the present invention provides a replacement organ formed or formable using a matrix according to the second or third aspects of the invention. The replacement organ may be, e.g., a replacement heart, kidney, liver, etc. The replacement organ may be an in vivo replacement organ, i.e. transplanted into a patient, or it may be an ex vivo organ, such as an organ assist device, to be located outside the body, such as a liver assist device.

In a sixth aspect, the present invention provides a bioreactor including a matrix according to any one of the second, third or fourth aspects disposed in a vessel, the bioreactor further including means for flowing cell culture medium along the vessel and through the matrix. Preferably, the vessel is the vessel in which the matrix was formed.

Typically, the bioreactor also includes means for flowing cell culture medium through the matrix.

In a preferred embodiment, the hardened matrix is formed in a vessel as described with respect to a preferred feature of the first aspect. This vessel preferably is part of the bioreactor, so that the hardened matrix need not be removed from the vessel before use in the bioreactor. This can maintain the sterility of the hardened matrix and improves the safety of the bioreactor.

The bioreactor typically comprises a chamber containing a culture of cells to which a flow of cell culture medium is supplied. The flow of medium may for example be continuous or intermittent.

The bioreactor may comprise one or more fluid inlets or outlets for supply of culture medium to the hardened matrix. Furthermore, it may comprise one or more ports for probes for measuring conditions such as pH, CO₂ content, oxygen content, etc. in the bioreactor. In a preferred embodiment, this bioreactor can support a flow of culture medium along the full length of and/or throughout the hardened matrix.

An important aspect of the bioreactor is that cell culture medium can flow from one end of the matrix to the other. In this sense, it is preferred that the voids within the matrix are interconnected, since this allows flow from one void to the next, promoting easy flow.

Another advantage of maintaining the hardened matrix in the vessel is that the matrix may be relatively delicate and sensitive to handling. Handling has the potential to damage the matrix itself or the cells to be cultured.

In a seventh aspect of the invention, there is provided a method of forming a predetermined shape of a hardened material including the steps of: contacting a hardenable liquid with a mould defining, at least in part, the predetermined shape, wherein a contacting surface of the mould includes a hardening agent; and allowing the hardenable liquid to harden by chemical interaction with the hardening agent to form the predetermined shape.

Preferably, the mould includes a vessel and at least one removable insert. Typically, the removable insert is removed by dissolving it or by partially dissolving it and mechanically removing it. The removable insert may be formed of similar materials as described with respect to the volume blanking structure of the first aspect.

This aspect of the invention is similar to the first aspect of the invention in the sense that the method may be used to give a desirable internal shape to a hardened material. In particular, this aspect of the invention may allow the formation of hardened materials with complex internal shapes, for example one or more internal space in the hardened material. These shapes may mimic the shape of body parts. For example, they may mimic the shape of vessels, valves (e.g. heart valves) or organs or parts of organs such as the heart, bones, etc.

The hardenable liquid is preferably the same as that used with respect to the first aspect. This aspect of the invention preferably includes any preferred feature as described with respect to any of the other aspects of the invention. In particular, a preferred embodiment of the invention combines the first and seventh aspect to give a method for producing a matrix of hardened material of predetermined shape.

A further aspect of the present invention provides a hardened matrix or a hardened material of predetermined shape obtained or obtainable by any of the methods of the previous aspects.

### INTRODUCTION OF THE DRAWINGS

Preferred embodiments of the present invention will now be described by way of example only, with reference to the accompanying drawings, in which:

FIG. 1 shows a schematic sectional view of a first embodiment of the present invention.

FIG. 2 shows a schematic view along the line A-A′ in FIG. 1.

FIG. 3 shows a schematic, modified, enlarged view of a part of the packing arrangement of FIG. 1.

FIG. 4 shows a schematic sectional view of a bead for use in an embodiment of the present invention.

FIG. 5 shows a schematic sectional view of a hardened matrix according to an embodiment of the present invention.

FIG. 6 shows a schematic sectional view of a second embodiment of the present invention.

FIG. 7 shows a schematic view along the line B-B′ in FIG. 6.

FIG. 8 shows a schematic sectional view of a bioreactor according to an embodiment of the present invention.

FIG. 9 shows a schematic sectional view of a hardened matrix according to another embodiment of the invention.

FIG. 10 is a sectional view of a hardened matrix formed according to another embodiment of the invention.

FIG. 11 is a sectional view of a hardened matrix formed according to another embodiment of the invention.

FIG. 12 is a sectional view of a forming apparatus for forming a hardened material in a predetermined shape according to another embodiment of the invention.

### DESCRIPTION OF PREFERRED EMBODIMENTS

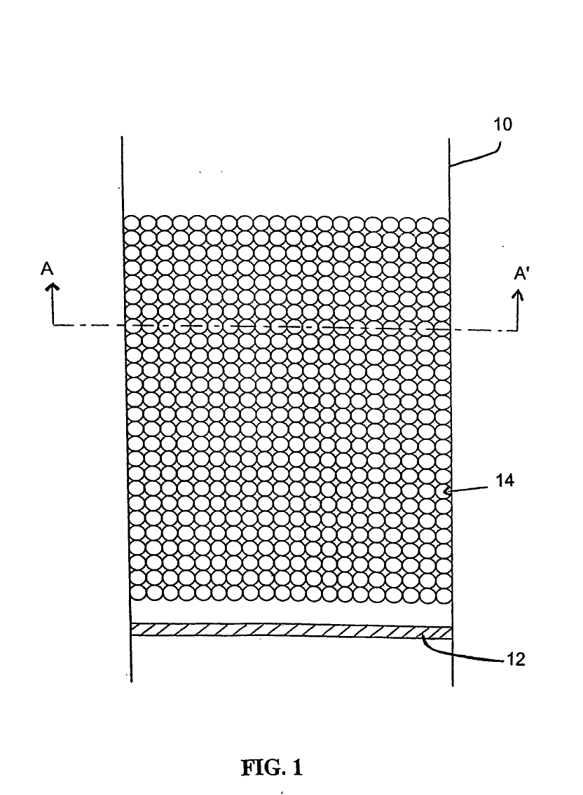

FIG. 1 shows a schematic sectional view of an apparatus for producing a hardened matrix according to an embodiment of the invention. In FIG. 1, a tubular vessel 10 has a sealing plate 12 located at its lower end. Sealing plate 12 is provided to ensure that liquid in the tube 10 above sealing plate 12 does not leak out of the lower end of tube 10. Sealing plate 12 is removable. It may, for example, be a plunger, capable of sliding upwards or downwards within the tube 10 (which may be a converted syringe).

An array of discrete beads 14 is packed within tube 10. The array of beads is an example of a volume blanking structure. It is to be noted here that the schematic packing arrangement as shown in FIG. 1 is very regular. In practical embodiments of the invention, it is likely that the packing of beads 14 will be more or less random. This is because beads 14 are typically small, for example around 50 μm in diameter. Beads 14 may be packed in tube 10 simply by pouring the beads into tube 10. More sophisticated alternate packing arrangements are described below.

As will be clear to the skilled person, the schematic square packing of beads 14 in FIG. 1 is unlikely to occur in practice. However, for now, this schematic arrangement serves an illustrative purpose.

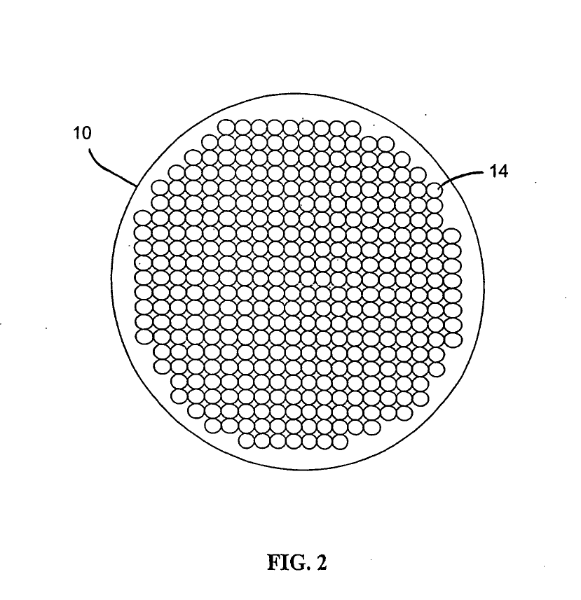

FIG. 2 shows a schematic view along line A-A′ in FIG. 1. This sectional view from above shows that the beads 14 substantially fill the vessel 10 in the width direction. If necessary, plate 12 supports the beads 14.

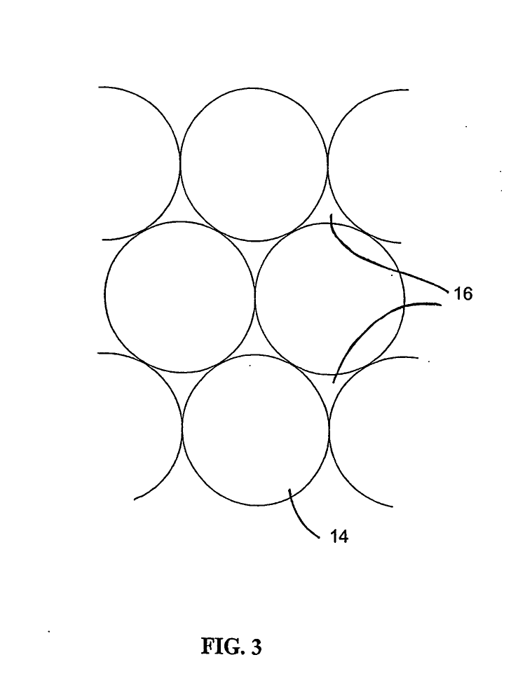

Beads 14 are typically of rounded shape, as illustrated in the drawings. Preferably, they are spherical. FIG. 3 shows a schematic enlarged view of some packed beads 14. It is to be noted here that, on a small scale, the beads 14 will tend to be relatively closely packed, as illustrated in FIG. 3. It is the long range order of the beads 14 which will tend to be relatively random. Between adjacent beads 14 are interstitial spaces 16. If the beads are approximately spherical (as in this embodiment), then interstitial spaces 16 are interconnected. Thus, the interstitial spaces in the packed beads arrangement define flow paths through the packed beads arrangement.

It is preferred that beads 14 are of similar size to each other. This gives rise to similarly sized interstitial spaces. However, of course, in practical embodiments, there will be some size distribution of beads 14. For this reason, there will be some size distribution of interconnected spaces 16.

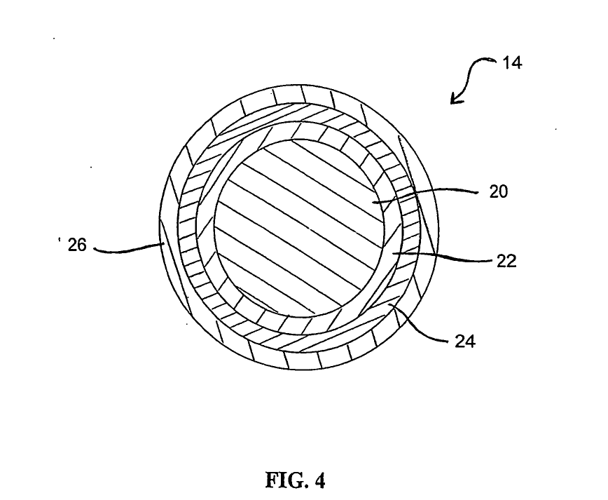

FIG. 4 shows a schematic sectional view of a typical bead 14. Bead 14 includes a core 20 of soluble material such as glucose. This has a coating 22 of a cell growth factor. On coating 22 is a layer 24 of hardening agent, in this case calcium chloride. The bead 14 has an outer protective coating 26.

Once the beads 14 have been packed within tubular vessel 10, a hardenable liquid (not shown) is poured into tubular vessel 10 to fill the spaces 16 between beads 14. The hardenable liquid in this case is alginate. A suitable volume of liquid is used such that there is little or no excess liquid above or below the packed beads arrangement.

It should be noted here that the beads could be poured into the hardenable liquid. The beads would then pack themselves into a self-supporting structure (helped by the vessel) and thus blank out the liquid from the volume occupied by the bead bodies.

Protective coating 26 on each bead dissolves at a predetermined rate in the hardenable liquid. This protective coating 26 prevents immediate exposure of the hardenable liquid to the hardening agent layer 24. Thus, the liquid has time to fill all the available spaces between beads 14. The protective layer 26 may have a dissolution rate dependent on the pH of the hardenable liquid. Thus, the pH of the hardenable liquid may be altered after pouring into the vessel (e.g. by adding suitable acidic or alkaline substances to the liquids) in order to trigger dissolution of protective layer 26. Of course, the allowable range of alteration of pH of the hardenable liquid will depend on the effect of pH on biological agents contained in the hardenable liquid.

Once the protective layer 26 has dissolved, the alginate comes into contact with the calcium chloride layer. This has the effect of rapidly hardening the alginate. Typically, the thickness of the calcium chloride layer is tailored to the volume of alginate which it is estimated will come into contact with bead 14.

Beads 14 may be made by known methods of spray forming. Initially, the glucose core 20 is formed and this is subsequently coated by layers 22, 24, 26 in a continuous process. Careful control of the spray forming conditions can lead to a uniform size distribution of beads 14 and also uniform distributions of thicknesses of layers 22, 24, 26. See, for example, New Approaches to Tablet Manufacture. Dr. Marshall Whiteman, Phoqus. European Pharmaceutical Review, Vol. 4, Issue 3, Autumn 1999, and Cowley M, 1999, Powder Coating: Assessment of component being coated: A practical guide to equipment, processes and productivity at a profit, pp. 13-31.

Once the alginate is hardened by cross-linking due to interaction with the calcium ions in the calcium chloride layer 24, the cell growth factor layer 22 is exposed within the hardened alginate matrix. This can be allowed to leach into the hardened alginate matrix as desired. This can lead to a desirable concentration gradient in growth factor concentration within the hardened alginate. Since the glucose core 20 is relatively benign in biological terms, the glucose core 20 can be allowed to remain in place for some time while the growth factor 22 leaches into the hardened alginate matrix.

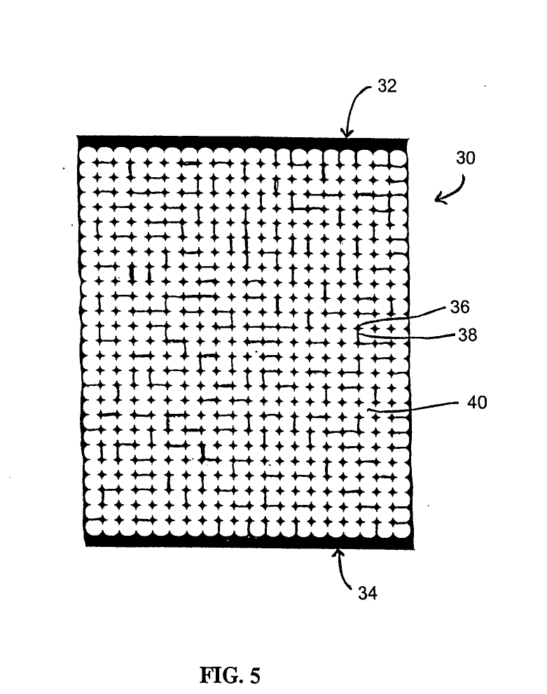

Subsequently, the glucose core 20 may be removed by passing water through the hardened alginate matrix to dissolve the glucose. Once the glucose core has been removed, the hardened alginate matrix contains voids where the beads 14 were located. A schematic hardened alginate matrix 30 is illustrated in FIG. 5. This is a sectional view. “Hardened” alginate is relatively soft and gel-like. The view shown in FIG. 5 shows upper 32 and lower 34 portions of solidified alginate not containing voids. The remainder of the alginate matrix consists of a network of interconnected hardened alginate nodes 36. Since the spaces 16 in the packed bead arrangement were interconnected, the hardened alginate is interconnected since this has replaced the spaces 16. Of course, a typical sectional view will not show all of the interconnections between the various hardened alginate nodes 36. However, some of these connections 38 are illustrated in FIG. 5.

Since, in the packed beads arrangement, the beads abut each other, the resultant voids 40 left by the beads 14 are interconnected with each other (since the alginate liquid occupied only the space not occupied by the beads 14). This gives a hardened matrix 30 with an advantageous structure. The interconnected voids 40 provide flow paths for, e.g. culture medium through the hardened matrix 30.

In this preferred embodiment, living cells from a patient such as a patient's liver cells are mixed with the alginate liquid. Alginate liquid is biocompatible with liver cells. It must be ensured, of course, that the protective coating 26 is made of a material which will not harm the liver cells in the alginate liquid. A uniform distribution of cells within the alginate liquid will give rise to a substantially uniform distribution of cells within the alginate liquid which will give rise to a substantially uniform distribution of cells within the hardened alginate matrix 30. The provision of growth factor in the hardened alginate matrix promotes the growth of the cells. Preferably, the cells are cultured to grow and produce extra cellular material. The alginate matrix may be slowly consumed during this process. In this way, the cells replace the alginate matrix with their own tissue scaffold. This can improve the rigidity and biocompatibility of the arrangement.

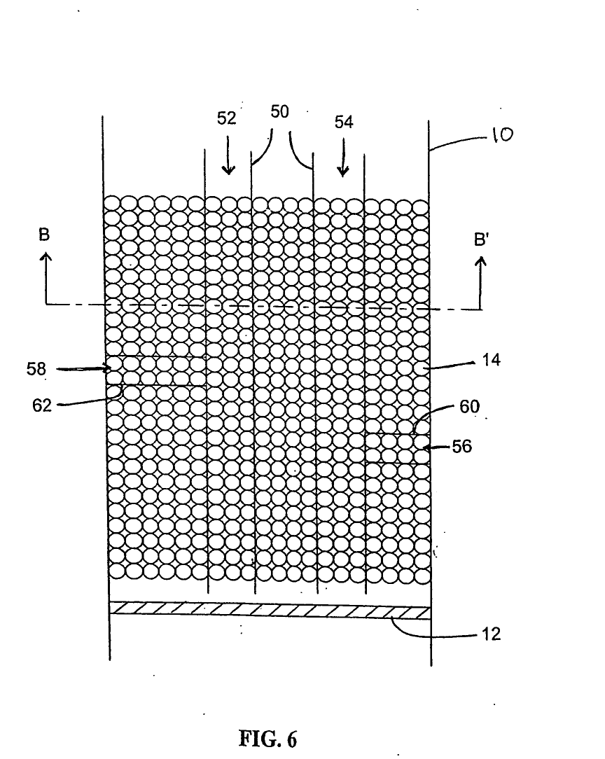

FIG. 6 shows a schematic sectional view of a further preferred embodiment of the present invention. FIG. 6 is similar to FIG. 1 in that is shows a tubular vessel 10 with a packed arrangement of beads 14 located above a sealing plate 12. These features will not be described in detail again.

The packing arrangement of beads 14 is more complex in FIG. 6 than in FIG. 1. In FIG. 6, regions of the packing are separated from the remainder of the packing arrangement by tubes formed from soluble films 50, extending downwards through the packing arrangement. These films 50 define square rod-shaped regions 52, 54 of packed beads which are isolated from the remainder of the packed beads. FIG. 6 also shows horizontal regions 56, 58 of similarly isolated beads, these being isolated by films 60, 62.

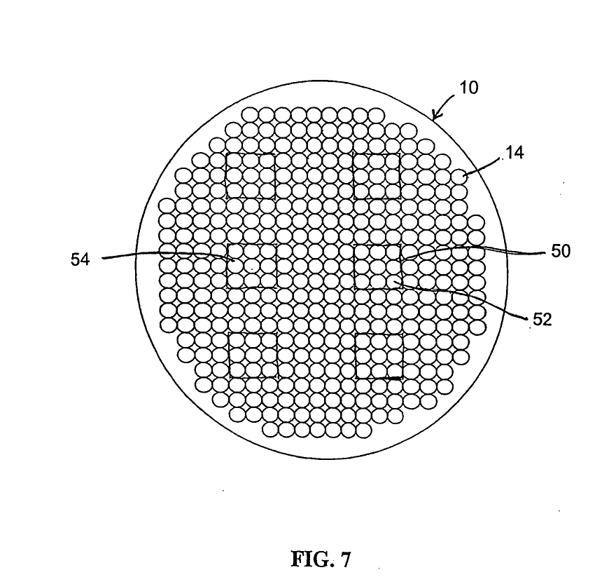

FIG. 7 shows a schematic view along line B-B′ in FIG. 6. This shows an array of vertically extending square rod-shaped regions which are isolated from the remainder of the packed bead arrangement.

Before or during packing of the main bead arrangement, isolation film 50, for example, is selectively packed with beads having no or little hardening agent layer 24. This film is nevertheless packed with beads in order to maintain its shape within the packed arrangement. Very thin, flexible films are used since these may be soluble. It would of course be possible to use rigid, empty (unpacked) tubes in the same role, but removal of these tubes from the hardened matrix may damage the matrix.

As will be clear, when the hardenable alginate liquid in poured into the vessel 10, the liquid occupies the spaces 16 between beads 14. The liquid is also poured down regions 52, 54. However, in these regions 52, 54 the alginate does not harden since there is no sufficient available hardening agent. Once the remainder of the alginate has hardened, the alginate in regions 52, 54 may be removed. Film 50 may then be dissolved away. This leaves a hardened alginate matrix containing vertically (and horizontally in the case of regions 56, 58) extending channels. In this way, complex internal shapes which mimic the shapes of organs such as the liver, heart, kidneys may be formed.

Furthermore, in alternative preferred embodiments, regions 52, 54 could be filled with beads containing alternative growth factors to the remainder of the beads. These regions may then be filled with an alginate liquid seeded with different cells to the cells seeded in the remainder of the alginate liquid used in the rest of the arrangement. In this way, complex, cell-differentiated structures may be engineered.

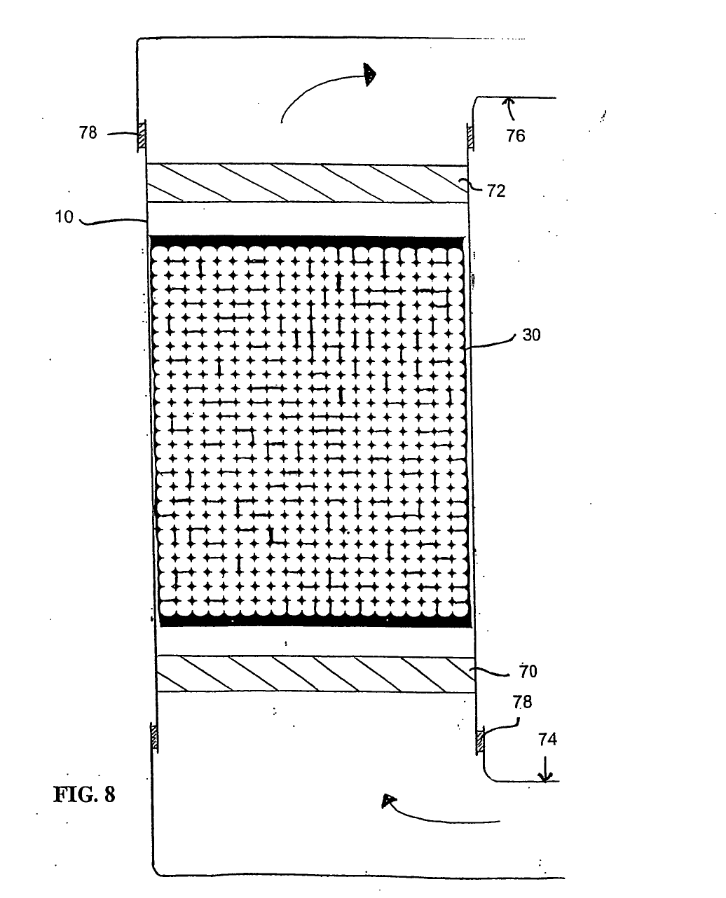

FIG. 8 shows a bioreactor according to a preferred embodiment of the present invention. In FIG. 8, an alginate matrix 30 has been formed within tubular vessel 10, as described above. This alginate matrix 30 is seeded with cells which can produce a useful biological agent. Hardened alginate matrix 30 is not removed from tubular vessel 10. Instead, sealing plate 12 has been removed. The upper and lower ends of tubular vessel 10 are filled by sinter plugs 70, 72. These are rigid yet porous plugs which will prevent movement of hardened alginate matrix 30 out of tube 10. The tube 10 is connected to a cell culture circuit (not shown complete) including cell culture input tube 74 and cell culture exhaust tube 76. These are connected to tubular vessel 10 via sealing member 78 (e.g. O-rings). In this way, the cells within the hardened alginate matrix may be grown and cultured and their products harvested without invasive and potentially non-sterile removal of alginate matrix 30 from tubular vessel 10.

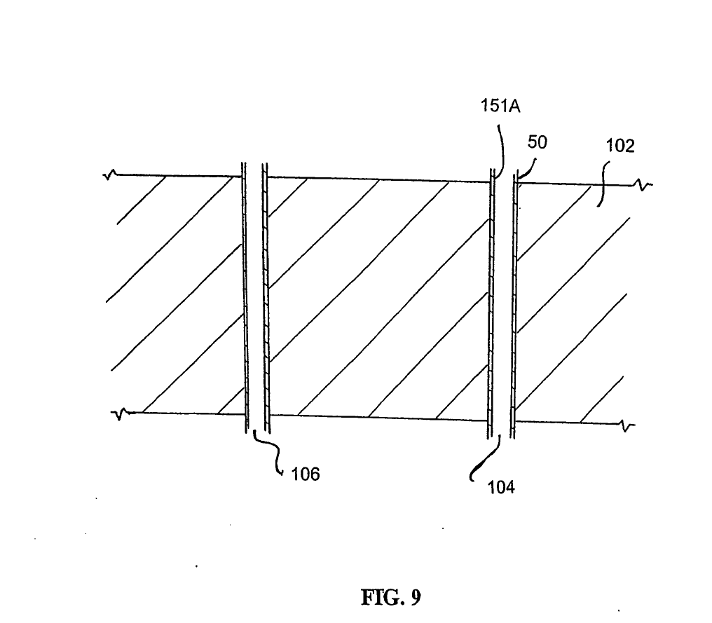

FIGS. 6 and 7 show elongate regions of approximately square cross-section. It is of course possible to make these elongate regions with rounded, e.g. circular cross-section. As has already been described, these regions can be formed so as to create tubular spaces in the hardened matrix. These tubular spaces may themselves be filled with an alternate hardened matrix. Alternatively, the internal surfaces of the tubular spaces may be coated with a hardened material. This is illustrated in FIG. 9, which shows a hardened matrix 102 with tubular spaces 104, 106 formed in it by the above-described method. The film 50, in this case, has not yet been dissolved away. The film 50 is formed on the internal surface of the tubular space. A hardened coating 151A is formed on the exposed surface of the film 50. This formation of coating 151A may be independent of the matrix, so that only the film 50, acting as a vessel, takes part in the formation of coating 151A.

Coating 151A is a hardened alginate, in the form of a tube. It may be formed in several different ways, as described below.

Methods and apparatuses suitable for forming a thin-walled tube of hardened material within the hardened matrix such as the tube 151A, are described and illustrated in WO-02/77336 mentioned above, particularly in FIGS. 1 to 4 and 8 to 14, to which reference should be made.

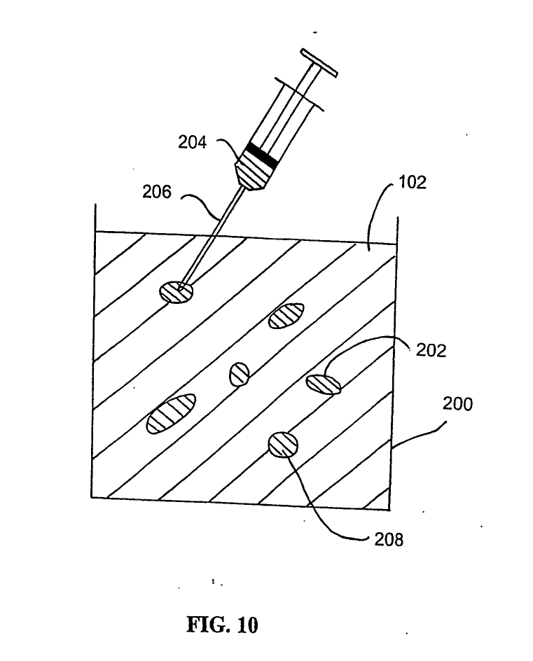

Another embodiment of the invention is illustrated in FIG. 10. This shows a hardened alginate matrix 102 formed within a vessel 200 in a way similar to the first embodiment. However, in this case, the volume blanking beads used had a bimodal size distribution. Most were small but a few were relatively large (around 5 mm). After dissolution of the beads, large pores 202 were left within the matrix 102, in addition to the smaller pores (not shown). The matrix 102 is seeded with cells of a first type by mixing with the hardenable liquid.

Large pores 202 are subsequently filled with a mixture 204 of beads and hardenable liquid, seeded with a second type of cells. These are injected into the large pores 202 via a needle 206. In this case, it is important that the beads include a protective layer to prevent the alginate from hardening immediately (i.e. before injection).

Mixture 204 hardens into secondary matrix 208. Thus, clumps of cells of a second type may be formed within a surrounding matrix seeded with cells of the first type.

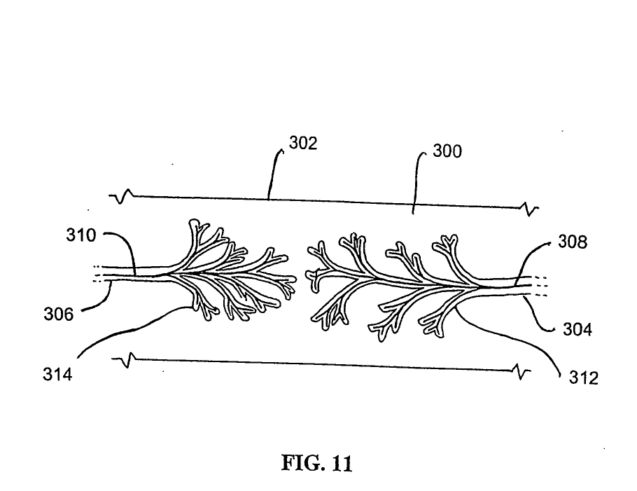

A further embodiment is illustrated in FIG. 11. This shows a hardened alginate matrix 300 formed substantially in accordance with the first embodiment within a tubular vessel 302. The matrix is formed around two tree-shaped inserts 304, 306. These are shaped with a similar external appearance to, e.g. branching blood vessels.

Inserts 304, 306 have an insoluble skeleton 308, 310, e.g. formed from biocompatible metal wire. On this skeleton is formed a coating 312, 314 of a soluble material. Inserts 304, 306 are removable from the hardened matrix (once hardened) by dissolving the coatings 312,314 and pulling the skeleton 308, 310. In this way, the hardened matrix may be formed with extremely complex shapes as, e.g. tissue growth scaffolds.

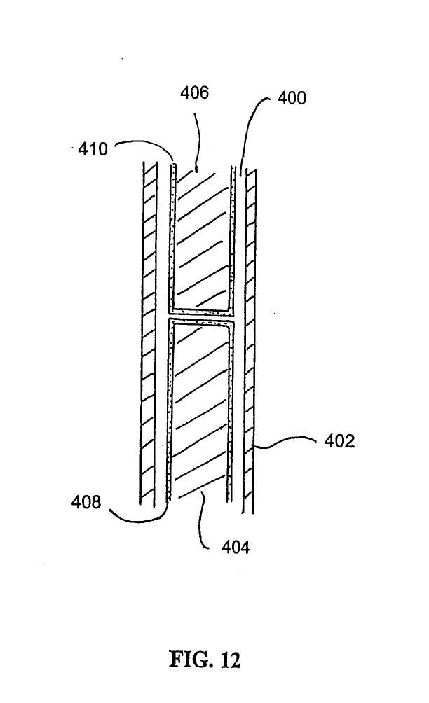

FIG. 12 illustrates another embodiment of the present invention. A predetermined shape 400 of hardened alginate material is formed in a mould consisting of a tubular vessel 402 and a pair of inserts 404, 406. The inserts take up a large proportion of the internal space defined by the tubular vessel 402. The space remaining is the predetermined shape mentioned above. Liquid alginate is fed into this space. Alternatively, the inserts may be pushed in after the liquid alginate in located in the vessel 402. Inserts 404, 406 each have a coating 408, 410 of calcium chloride. This contacts the liquid alginate and allows it to harden. Subsequently, the inserts 404, 406 are removed and the predetermined shape of hardened alginate is removed from the vessel 402.

The shape illustrated mimics (schematically) the shape of a heart valve. The hardened alginate here is a heart valve tissue engineering scaffold. In this embodiment, since the shape has thin walls, there is no need to include beads to harden the alginate through its thickness, or to leave voids.

Only simple apparatus is required to put the present invention into practice. Sterile, single-use, disposable apparatus suitable for practising the methods described can be readily produced at low cost. Manipulations of cells and formation of structures according to the present invention can thus be performed under sterile conditions at minimum expense and with minimum risk of contamination. Because of the simplicity of the apparatus required, the methods described herein can easily be automated.

The above embodiments have been described by way of example only. Modifications of these embodiments, further embodiments and modifications thereof will be apparent to the skilled person and as such are within the scope of the present invention.

1. A method of forming a matrix of hardened material, including the steps of:
contacting a hardenable liquid with a volume blanking structure, the structure having a dispersion of interconnected spaces therein and including a hardening agent, whereby the hardenable liquid occupies at least some of said spaces in said structure; and
allowing the hardenable liquid to harden by interaction with the hardening agent to form the matrix.

2. A method according to claim 1 wherein the volume blanking structure is formed before the hardenable liquid is contacted with said structure.

3. A method according to claim 1 wherein the hardening interaction is a chemical interaction.

4. A method according to claim 1 further including the step of removing the volume blanking structure to leave corresponding voids in the matrix of hardened material.

5. A method according to claim 4 wherein the volume blanking arrangement is removed by dissolving it.

6. A method according to claim 1 wherein the matrix of hardened material is a bulk matrix.

7. A method according to claim 6 wherein the bulk matrix has a three-dimensional shape and wherein the smallest dimension is not less than 0.5, 1, 2, 5 or 10 mm.

8. A method according to claim 1 including the step of distributing or seeding a bioactive agent in the matrix.

9. A method according to claim 8 wherein the bioactive agent is dispersed in the hardenable liquid.

10. A method according to claim 1 wherein the hardened matrix is biocompatible.

11. A method according to claim 1 wherein the volume blanking structure is an arrangement of volume blanking elements and the interconnected spaces are interstices between adjacent volume blanking elements.

12. A method according to claim 11 wherein the volume blanking elements are packed so that adjacent elements touch.

13. A method according to claim 11 wherein the volume blanking elements have a size in the range 1-100 μm.

14. A method according to claim 11 wherein each volume blanking element is a bead.

15. A method according to claim 14 wherein each bead is approximately spherical in shape.

16. A method according to claim 11 wherein the hardening agent is formed as a layer on at least some of the volume blanking elements.

17. A method according to claim 16 wherein the hardening agent has a protective layer formed over it which dissolves and delays the exposure of the hardening agent to the hardenable liquid.

18. A method according to claim 17 wherein the solubility of the protective layer is dependent on pH and the dissolution of the protective layer is triggered by a change in pH of the hardenable liquid.

19. A method according to claim 16 wherein the hardening agent layer has a cell growth factor layer under it.

20. A method according to claim 1 wherein the formation of the volume blanking structure includes the formation of one or more selected regions within the structure with different concentrations of hardening agent to the remainder of the structure.

21. A method according to claim 20 wherein each selected region is an elongate region extending through the structure.

22. A method according to claim 20 wherein each selected region is separated from the remainder of the structure by a retaining surface.

23. A method according to claim 22 wherein the retaining surface is a surface of a soluble film.

24. A method according to claim 20 wherein the concentration of hardening agent in each selected region is insufficient to harden the hardenable liquid placed in the spaces in that region.

25. A method according to claim 1 wherein the hardenable liquid is alginate.

26. A method according to claim 1 wherein the hardening agent includes calcium ions.

27. A method according to claim 1 wherein the volume blanking arrangement includes glucose.

28. A matrix of hardened material obtained via the method of claim 1.

29. A matrix of hardened material obtainable via the method of claim 1.

30. A body having a matrix of biocompatible material hardened in vitro by chemical interaction and an array of interconnected voids, the matrix of hardened material having a controlled distribution of a bioactive agent within it, and wherein the body is not a sheet or tube.

31. A body according to claim 30 wherein the bioactive agent is a pharmaceutical or other bioactive molecule, e.g. a pharmaceutical, enzyme, growth factor, hormone, cytokine, antibody, or nucleic acid, to be delivered to a desired site in a living mammal.

32. A body having a matrix of biocompatible in vitro hardened material and an array of interconnected voids, the matrix of hardened material having a distribution of bioactive agent in the form of cells within it, and wherein the body is not a sheet or tube.

33. A body according to claim 30 wherein the array of interconnected voids is in the form of a packed structure of contacting rounded shapes, such as spheres.

34. A body according to claim 30 wherein the interconnected voids are partially separated from each other by nodes of hardened material, each node having a controlled distribution of the bioactive agent through its thickness.

35. A body according to claim 30 wherein the bioactive agent is viable cells, killed cells or isolated cellular organelles.

36. A tissue growth scaffold including a matrix according to claim 28.

37. A method of tissue growth, e.g. organ production, comprising culturing of cells contained within the hardened material of the matrix according to claim 28 and/or culturing of cells contained in the pores of any such matrix.

38. Tissue, e.g. an organ, grown by the method of claim 37.

39. A bioreactor including the matrix according to claim 28 disposed in a vessel, the bioreactor further including means for flowing cell culture medium along the vessel and through the matrix.

40. A bioreactor according to claim 39 wherein the vessel is the vessel in which the matrix was formed.

41. A method of forming a predetermined shape of a hardened material including the steps of:
contacting a hardenable liquid with a mould defining, at least in part, the predetermined shape, wherein a contacting surface of the mould includes a hardening agent; and
allowing the hardenable liquid to harden by chemical interaction with the hardening agent to form the predetermined shape.

42. A method according to claim 41 wherein the mould includes a vessel and at least one removable insert.

43. A method according to claim 42 wherein the removable insert is removed by dissolving it or by partially dissolving it and mechanically removing it.
